(19) United States
(12) Patent Application Publication
(10) Pub. No.: US 2026/0048098 A1
(43) Pub. Date: Feb. 19, 2026

(54) TREATMENT OF RETINAL GANGLION CELL LOSS

(71) Applicant: PS Therapy, Inc., Westmont, IL (US)

(72) Inventor: Siddharth Narendran, Tamil Nadu (IN)

(21) Appl. No.: 19/362,976

(22) Filed: Oct. 20, 2025

(30) Foreign Application Priority Data
Aug. 18, 2024 (IN) 202441079163

(51) Int. Cl. A61K 38/17; A61K 45/06

(52) U.S. Cl. CPC A61K 38/1767; A61K 45/06

(57) ABSTRACT

The present invention is directed to methods of treating or preventing glaucoma comprising administering an effective amount of one or more compounds selected from the group consisting of transient receptor potential vanilloid 4 (TRPV4) antagonists, Grammostola spatulata MTx4 peptide and D-Grammostola spatulata MTx4 peptide to a subject in need thereof.

The present invention is further directed to methods of treating or preventing retinal ganglion cell loss comprising administering an effective amount of one or more compounds selected from the group consisting of transient receptor potential vanilloid 4 (TRPV4) antagonists, Grammostola spatulata MTx4 peptide and D-Grammostola spatulata MTx4 peptide to a subject in need thereof.

### FIELD OF THE INVENTION

The present invention is directed to methods of treating or preventing glaucoma comprising administering an effective amount of one or more compounds selected from the group consisting of transient receptor potential vanilloid 4 (TRPV4) antagonists, Grammostola spatulata MTx4 peptide and D-Grammostola spatulata MTx4 peptide to a subject in need thereof.

The present invention is further directed to methods of treating or preventing retinal ganglion cell loss comprising administering an effective amount of one or more compounds selected from the group consisting of transient receptor potential vanilloid 4 (TRPV4) antagonists, Grammostola spatulata MTx4 peptide and D-Grammostola spatulata MTx4 peptide to a subject in need thereof.

### BACKGROUND OF THE INVENTION

Glaucoma affects more than 78 million people worldwide. Glaucoma most often occurs in individuals over the age of 60. As the worldwide population of individuals over the age of 60 is set to double glaucoma is expected to become an even larger issue.

Glaucoma is a multifactorial disease which encompasses a spectrum ranging from elevated intraocular pressure (IOP) to reduced vascular perfusion of the optic nerve and progressive optic nerve damage. Glaucoma often leads to blindness.

A primary risk factor for glaucoma is elevated intraocular pressure (IOP), which results from impaired aqueous humor drainage. One of the key pathological features of glaucoma is the loss of retinal ganglion cells (RGCs), which are the primary neurons responsible for transmitting visual information from the retina to the brain. Currently, the available therapeutic options for glaucoma are limited and primarily focus on lowering IOP. Thus, there remains a need in the art for therapeutic treatment of retinal ganglion cell loss due to glaucoma.

### SUMMARY OF THE INVENTION

The present invention is directed to methods of treating or preventing glaucoma comprising administering an effective amount of one or more compounds selected from the group consisting of transient receptor potential vanilloid 4 (“TRPV4”) antagonists, Grammostola spatulata MTx4 peptide and D-Grammostola spatulata MTx4 peptide to a subject in need thereof.

The present invention is further directed to methods of treating or preventing retinal ganglion cell loss comprising administering an effective amount of one or more compounds selected from the group consisting of TRPV4 antagonists, Grammostola spatulata MTx4 peptide and D-Grammostola spatulata MTx4 peptide to a subject in need thereof.

### DETAILED DESCRIPTION OF THE INVENTION

The Applicant has surprisingly discovered that retinal ganglion cell loss associated with glaucoma can be treated or prevented by administering an effective amount of one or more compounds selected from the group consisting of transient receptor potential vanilloid 4 (“TRPV4”) antagonists, Grammostola spatulata MTx4 peptide and D-Grammostola spatulata MTx4 peptide.

In one embodiment, the present invention is directed to methods of treating or preventing glaucoma comprising administering an effective amount of one or more compounds selected from the group consisting of TRPV4 antagonists, Grammostola spatulata MTx4 peptide and D-Grammostola spatulata MTx4 peptide to a subject in need thereof.

In another embodiment, the present invention is further directed to methods of treating or preventing retinal ganglion cell loss comprising administering an effective amount of one or more compounds selected from the group consisting of TRPV4 antagonists, Grammostola spatulata MTx4 peptide and D-Grammostola spatulata MTx4 peptide to a subject in need thereof.

Administration of the compounds and compositions of the present invention may occur orally, topically, intravitreal, periocular, systemic absorption, as an injection or via an injectable implant. Compounds of the present invention may be formulated in a drop, gel, cream, ointment, suspension, an injectable solution and the like.

Grammostola spatulata MTx4 (CAS #1209500-46-8), also known GsMTx4 has the following chemical structure:

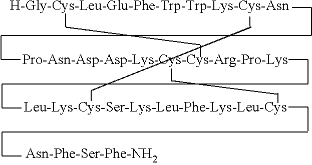

wherein “Gly” denotes glycine, “Cys” denotes cysteine, “Leu” denotes leucine, “Glu” denotes glutamate, “Phe” denotes phenylalanine, “Trp” denotes tryptophan, “Lys” denotes lysine, “Asn” denotes asparagine, “Pro” denotes proline, “Asp” denotes aspartate, “Arg” denotes arginine, and “Ser” denotes serine.

D-Grammostola spatulata MTx4 is the D isomer of Grammostola spatulata MTx4.

TRPV4 antagonists include, but are not limited to, GSK2193874 (CAS #1336960-13-4), GSK2798745 (CAS #1419609-94-1), 5,6-dichloro-2-(isopropylamino)-N,N-dimethyl-1-(1-phenylpiperidin-4-yl)-1H-benzo[d]imidazole-4-carboxamide, 5,6-Dichloro-2-(isopropylamino)-N,N-dimethyl-1-((1-phenylpiperidin-4-yl)methyl)-1H-benzo[d]imidazole-4-carboxamide, 5,6-dichloro-2-(isopropylamino)-N,N-dimethyl-1-(4-piperidin-1-yl)phenyl)-1H-benzo[d]imidazole-4-carboxamide, 1-(1-benzylpiperidin-4-yl)-5,6-dichloro-2-(isopropylamino)-N,N-dimethyl-1H-benzo[d]imidazole-4-carboxamide, 5,6-dichloro-1-(1-((4-chlorophenyl) sulfonyl) piperidin-4-yl)-2-(isopropylamino)-N,N-dimethyl-1H-benzo[d]imidazole-4-carboxamide, 5,6-dichloro-2-(isopropylamino)-N,N-dimethyl-1-(1-(phenylcarbamoyl) piperidin-4-yl)-1H-benzo[d]imidazole-4-carboxamide, 1-(1-(4-(tert-butyl)benzoyl) piperidin-4-yl)-5,6-dichloro-2-(isopropylamino)-N,N-dimethyl-1H-benzo[d]imidazole-4-carboxamide, 5,6-dichloro-2-(isopropylamino)-N,N-dimethyl-1-(1-(meta-tolyl) piperidin-4-yl)-1H-benzo[d]imidazole-4-carboxamide, 5,6-dichloro-2-(isopropylamino)-N,N-dimethyl-1-(1-(para-tolyl) piperidin-4-yl)-1H-benzo[d]imidazole-4-carboxamide, 1-(1-(4-(tert-butyl)phenyl) piperidin-4-yl)-5,6-dichloro-2-(isopropylamino)-N,N-dimethyl-1H-benzo[d]imidazole-4-carboxamide, 5,6-dichloro-1-(1-(4-cyclohexylphenyl) piperidin-4-yl)-2-(isopropylamino)-N,N-dimethyl-1H-benzo[d]imidazole-4-carboxamide, 5,6-dichloro-1-(1-(4-isopropoxyphenyl) piperidin-4-yl)-2-(isopropylamino)-N,N-dimethyl-1H-benzo[d]imidazole-4-carboxamide, methyl 2-(4-(4-(5,6-dichloro-4-(dimethylcarbamoyl)-2-(isopropylamino)-1H-benzo[d]imidazol-1-yl) piperidin-1-yl)phenyl)-2-methylpropanoate, 2-(4-(4-(5,6-dichloro-4-(dimethylcarbamoyl)-2-(isopropylamino)-1H-benzo[d]imidazol-1-yl) piperidin-1-yl)phenyl)-2-methylpropanoic acid, 5,6-dichloro-1-(1-(4-(2-cyanopropan-2-yl)phenyl) piperidin-4-yl)-2-(isopropylamino)-N,N-dimethyl-1H-benzo[d]imidazole-4-carboxamide, 5,6-dichloro-1-(1-(4-(4-cyanotetrahydro-2H-pyran-4-yl)phenyl) piperidin-4-yl)-2-(isopropylamino)-N,N-dimethyl-1H-benzo[d]imidazole-4-carboxamide, 2,4-Dichloro-N-isopropyl-N-(2-isopropylaminoethyl)benzenesulfonamide (CAS #946387-07-01), 2-Methyl-1-(3-morpholinopropyl)-5-phenyl-N-(3-(trifluoromethyl)phenyl)-1H-pyrrole-3-carboxamide.

The compounds of the present invention can be used in the form of pharmaceutically acceptable salts derived from inorganic or organic acids or bases. The phrase “pharmaceutically acceptable salt” means those salts which are, within the scope of sound medical judgment, suitable for use in contact with the tissues of humans and lower animals without undue toxicity, irritation, allergic response and the like and are commensurate with a reasonable benefit/risk ratio. Pharmaceutically acceptable salts are well-known in the art. For example, S. M. Berge et al. describe pharmaceutically acceptable salts in detail in J. Pharmaceutical Sciences, 1977, 66:1 et seq.

The salts can be prepared in situ during the final isolation and purification of the compounds of the invention or separately by reacting a free base function with a suitable organic acid. Representative acid addition salts include, but are not limited to acetate, adipate, alginate, citrate, aspartate, benzoate, benzenesulfonate, bisulfate, butyrate, camphorate, camphorsulfonate, digluconate, glycerophosphate, hemisulfate, heptanoate, hexanoate, fumarate, hydrochloride, hydrobromide, hydroiodide, 2-hydroxyethansulfonate (isothionate), lactate, maleate, methanesulfonate, nicotinate, 2-naphthalenesulfonate, oxalate, palmitoate, pectinate, persulfate, 3-phenylpropionate, picrate, pivalate, propionate, succinate, tartrate, thiocyanate, phosphate, glutamate, bicarbonate, p-toluenesulfonate and undecanoate. Also, the basic nitrogen-containing groups can be quaternized with such agents as lower alkyl halides such as methyl, ethyl, propyl, and butyl chlorides, bromides and iodides; dialkyl sulfates like dimethyl, diethyl, dibutyl and diamyl sulfates; long chain halides such as decyl, lauryl, myristyl and stearyl chlorides, bromides and iodides; arylalkyl halides like benzyl and phenethyl bromides and others. Water or oil-soluble or dispersible products are thereby obtained. Examples of acids which can be employed to form pharmaceutically acceptable acid addition salts include such inorganic acids as hydrochloric acid, hydrobromic acid, hyaluronic acid, malic acid, sulphuric acid and phosphoric acid and such organic acids as oxalic acid, malic acid, maleic acid, methanosulfonic acid, succinic acid and citric acid.

Basic addition salts can be prepared in situ during the final isolation and purification of compounds of this invention by reacting a carboxylic acid-containing moiety with a suitable base such as the hydroxide, carbonate or bicarbonate of a pharmaceutically acceptable metal cation or with ammonia or an organic primary, secondary or tertiary amine. Pharmaceutically acceptable salts include, but are not limited to, cations based on alkali metals or alkaline earth metals such as lithium, sodium, potassium, calcium, magnesium and aluminum salts and the like and nontoxic quaternary ammonia and amine cations including ammonium, tetramethylammonium, tetraethylammonium, methylammonium, dimethylammonium, trimethylammonium, triethylammonium, diethylammonium, and ethylammonium among others. Other representative organic amines useful for the formation of base addition salts include ethylenediamine, ethanolamine, diethanolamine, piperidine, piperazine and the like.

In another preferred embodiment, the compositions of the present invention are oral compositions.

Oral compositions of the present invention may further comprise oils, fatty acids, vitamins and minerals.

Oils suitable for use in oral compositions of the present invention include, but are not limited to, fish oil, flaxseed oil and the like.

Fatty acids suitable for use in oral compositions of the present invention include, but are not limited to, omega-3 fatty acids such as linolenic acid, eicosapentaenoic acid, docosahexaenoic acid and the like.

Vitamins suitable for use in oral compositions of the present invention include, but are not limited to, vitamin A, beta carotene, vitamin B2, vitamin B12, vitamin C, vitamin E and the like.

Minerals suitable for use in oral compositions of the present invention include, but are not limited to, zinc, selenium, copper, manganese and the like and salts thereof.

As used herein the term “treatment” or “treating” refers to slowing the progression of or ameliorating glaucoma or one or more symptoms of glaucoma or slowing the progression of or ameliorating retinal ganglion cell loss.

As used herein the term “effective amount” refers to the amount necessary to treat a patient in need thereof.

Throughout the application, the singular forms “a,” “an,” and “the” include plural reference unless the context clearly dictates otherwise.

Throughout the application, all disclosed ranges include all possible values within those ranges. All possible values within the ranges disclosed in the application can also be used as endpoints for additional ranges between these values.

The disclosed embodiments are simply exemplary embodiments of the inventive concepts disclosed herein and should not be considered as limiting unless the claims expressly state otherwise.

The following example is intended to illustrate the present invention and to teach one of ordinary skill in the art how to use the methods of the invention. The example is not intended to be limiting in any way.

### EXAMPLE

### Example 1—Treatment of Retinal Ganglion Cell Loss (Prophetic)

### Method

A silicone oil-induced ocular hypertensive mouse model simulates retinal ganglion cell loss. These model mice were treated with TRPV4 antagonists, GsMTx4 peptide and/or D-GsMTx4 peptide. Following treatment, the mouse model exhibited less ganglion cell loss. Thus, it is evident that TRPV4 antagonists, GsMTx4 peptide and/or D-GsMTx4 peptide can be effectively used in controlling retinal ganglion cell loss.

What is claimed is:

1. A method of treating or preventing glaucoma comprising administering an effective amount of one or more compounds selected from the group consisting of transient receptor potential V4 (TRPV4) antagonists, Grammostola spatulata MTx4 peptide and D-Grammostola spatulata MTx4 peptide to a subject in need thereof.

2. The method of claim 1, wherein retinal ganglion cell loss is reduced or prevented.

3. The method of claim 1, wherein the TRPV4 antagonists are selected from the group consisting of.

4. The method of claim 1, wherein administration occurs orally, topically, as an injection or via an injectable implant.

5. The method of claim 1, wherein the mixture is administered topically in a composition comprising one or more excipients selected from the group consisting of carriers, surfactants, viscosity enhancers, tonicity adjustors, preservatives, antioxidants and buffers.

6. The method of claim 1, wherein the mixture is administered orally in a composition comprising one or more excipients selected from the group consisting of oils, fatty acids, vitamins and minerals.

7. A method of treating or preventing retinal ganglion cell loss comprising administering an effective amount of one or more compounds selected from the group consisting of transient receptor potential V4 (TRPV4) antagonists, Grammostola spatulata MTx4 peptide and D-Grammostola spatulata MTx4 peptide to a subject in need thereof.

8. The method of claim 7, wherein the subject has glaucoma.

9. The method of claim 7, wherein the TRPV4 antagonists are selected from the group consisting of.

10. The method of claim 7, wherein administration occurs orally, topically, as an injection or via an injectable implant.

11. The method of claim 7, wherein the mixture is administered topically in a composition comprising one or more excipients selected from the group consisting of carriers, surfactants, viscosity enhancers, tonicity adjustors, preservatives, antioxidants and buffers.

12. The method of claim 7, wherein the mixture is administered orally in a composition comprising one or more excipients selected from the group consisting of oils, fatty acids, vitamins and minerals.
